(19) United States
(12) Patent Application Publication
(10) Pub. No.: US 2021/0378389 A1
(43) Pub. Date: Dec. 9, 2021

(54) DEVICE FOR PROVIDING SERIAL SKIN TYPE ANALYSIS AND PREPARING CUSTOMIZED COSMETIC PRODUCT

(71) Applicant: Human Skin Clinical Trial Center, Seoul (KR)

(72) Inventors: Won Kyu Hong, Seoul (KR); Hong Seok Kim, Chungcheongbuk-do (KR); Hui Jeong Jeong, Gyeonggi-do (KR)

(73) Assignee: Human Skin Clinical Trial Center, Seoul (KR)

(21) Appl. No.: 17/337,872

(22) Filed: Jun. 3, 2021

(30) Foreign Application Priority Data
Jun. 8, 2020 (KR) 10-2020-0068940

(51) Int. Cl. A45D 44/00; A61B 5/00

(52) U.S. Cl. CPC A45D 44/005; A45D 2044/007; A61B 5/441

(57) ABSTRACT

Disclosed is a device for providing serial skin type analysis and preparing a customized cosmetic product. The device includes a measurer configured to measure a skin condition of a user, a storage unit configured to store data of the skin condition measured by the measurer, a determiner configured to index the measured skin condition data and determine an effective cosmetic ingredient based on the skin condition, and a transmitter configured to transmit the skin condition data.

### CROSS-REFERENCE TO RELATED APPLICATION

This application claims under 35 U.S.C. §119(a) the benefit of priority to Korean Patent Application No. 10-2020-0068940, filed on Jun. 8, 2020, the entire contents of which are incorporated herein by reference.

### BACKGROUND

### (a) Technical Field

The present invention relates to a device for providing serial skin type analysis and preparing a customized cosmetic product, and more particularly, a device for providing serial skin type analysis and preparing a customized cosmetic product that is capable of indexing a skin condition measured by a measurer and recommending effective cosmetic ingredients based on the indexed skin condition.

### (b) Background Art

Skin is largely divided into three layers, namely the epidermis, dermis, and subcutaneous fat tissue sequentially from the outside, and functions to protect the human body from foreign physical and chemical harm, and functions in particular to prevent moisture, which accounts for about 65% to about 70% of humans, from evaporating to the outside. In particular, the epidermis is divided into the stratum corneum, stratum granulosum, stratum spinosum, and stratum basale sequentially from the outside. The stratum corneum of the epidermis contains about 10 to about 20% moisture, is the outermost part of the human body, and functions to inhibit evaporation of water from the body while blocking excessive penetration of foreign substances (J. Invest. Dermatol. 80 (Suppl), 44-49 1983). The cells constituting the stratum corneum contain a high concentration of natural moisturizing factor (NMF), which is a water-soluble component that acts to impart flexibility and adequate moisture content to the skin (J. Invest. Dermatol. 54, 24-31, 1970).

However, the individual difference in internal factors determining skin characteristics, such as skin temperature and thickness, may cause individual differences in skin characteristics such as skin moisture and/or oil contents, elasticity and roughness. External factors such as environmental changes, lifestyle changes, social stresses, environmental pollution, frequent makeup removal, and natural skin aging over time may decrease the amount of moisture in the stratum corneum, thus resulting in skin dryness and roughened surface of the skin and thus loss of glossiness from the skin and dullness (darkness). In addition, internal factors such as individual skin characteristics may cause differences in the influence of external factors on the skin.

Therefore, a method of determining skin characteristics and selecting cosmetics containing ingredients suitable for the skin characteristics is required in order to fundamentally detect the skin data of users and improve the skin condition thereof.

The above information disclosed in this Background section is only for enhancement of understanding of the background of the invention, and therefore it may contain information that does not form the prior art that is already known in this country to a person of ordinary skill in the art.

### PRIOR ART

### PARENT DOCUMENT

(Parent Document 1) Utility Model Application No. 20-2010-0012450 (filed on Dec. 2, 2010)

### SUMMARY OF THE DISCLOSURE

The present invention has been made in an effort to solve the above-described problems associated with the prior art, and it is one object of the present invention to provide a device for providing serial skin type analysis and preparing a customized cosmetic product that is capable of comprehensively measuring the skin condition of a user.

It is another object of the present invention to analyze the skin data of a user and inform the user of effective cosmetic ingredients based on the skin condition.

The objects of the present invention are not limited to those described above. Other objects of the present invention will be clearly understood from the following description and embodiments of the present invention, and are able to be implemented by means defined in the claims and combinations thereof.

In an aspect, the present invention provides a device for providing serial skin type analysis and preparing a customized cosmetic product including the components described below.

Other aspects and preferred embodiments of the invention are discussed infra.

### BRIEF DESCRIPTION OF THE DRAWINGS

The above and other features of the present invention will now be described in detail with reference to certain exemplary embodiments thereof, illustrated in the accompanying drawings which are given hereinbelow by way of illustration only, and thus are not limitative of the present invention, and wherein:

FIG. 1 is a block diagram illustrating a device for providing serial skin type analysis and preparing a customized cosmetic product.

### DETAILED DESCRIPTION

Hereinafter, preferred embodiments of the present invention will be described in more detail with reference to the attached drawings. The embodiments may be modified in different forms and should not be construed as limiting the scope of the present invention. The embodiments are provided only to offer more complete description of the present invention to those skilled in the art.

In addition, terms such as “ . . . unit”, “ . . . unit”, and “ . . . module” described herein indicate components that perform at least one function or operation, and can be implemented as hardware, software, or a combination of hardware and software.

The terms “first”, “second”, etc. may be used only to distinguish one element from another element because the names of the elements modified by the terms “first”, “second”, etc. are otherwise identical. There is no limitation as to the order of the terms in the following description. Hereinafter, embodiments will be described in detail with reference to the accompanying drawings. Like reference numbers refer to identical or corresponding elements throughout the description of the figures, and redundant descriptions thereof will be omitted.

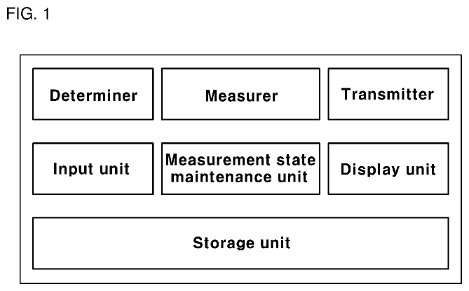

FIG. 1 shows a device for providing serial skin type analysis and preparing a customized cosmetic product according to the present invention, which is located inside a housing and is configured to measure a skin condition using a measurer and to output data pertaining to the skin condition.

The device for providing serial skin type analysis and preparing a customized cosmetic product may include a housing in which a measurer, a storage unit, a determiner and a transmitter are located, and the housing may be portable or fixed to a predetermined position.

The measurer located in the housing is configured to face the face of a user to be analyzed, and is configured to image skin data of the user using a plurality of devices located in the measurer.

More preferably, the measurer is configured to measure at least one of user's skin moisture content, skin barrier, skin pigmentation, skin erythema, skin wrinkles, or skin elasticity, and the determiner is configured to index data of the measured moisture content, skin barrier, skin pigmentation, skin erythema, skin wrinkles and skin elasticity, and to store the resulting indexes in the storage unit.

The index of each of the measured moisture content, skin barrier, skin pigmentation, skin erythema, skin wrinkles and skin elasticity may be determined within a range of 1 to 10. As the index reaches 10, the corresponding measurement value is determined to be high in the analyzed skin.

In an embodiment of the present invention regarding indexing of the data of the measured user's skin condition, (1) for oil content, 1-3 is dry, 4-7 is neutral, and 8-10 is oily, (2) for moisture content, 1-3 is lacking moisture, 4-7 is normal, and 8-10 is excessive moisture (skin condition like eczema), (3) for skin barrier index, 1-3 is normal, 4-7 indicates barrier damage, and 8-10 indicates excessive barrier damage and suspicion of skin disease, (4) for skin pigmentation index, 1-3 is light skin, 4-7 is moderate skin tone, and 8-10 is dark skin, (5) for skin erythema index, 1-3 is normal, 4-7 is slightly red skin, and 8-10 is excessively red skin, (6) for skin wrinkle index, 1-3 is few wrinkles, 4-7 is moderately wrinkled, and 8-10 is excessively wrinkled, and (7) for skin elasticity index, 1-3 is elastic skin, 4-7 is skin with reduced elasticity, and 8-10 is skin with excessively reduced elasticity.

Furthermore, the determiner is configured to determine a cosmetic ingredient matching the corresponding data among the plurality of indexes of the skin condition data.

In an embodiment of the present invention, the measurer acquires a skin image using an imaging device including a sensor array integrated with a capacitive sensor so as to acquire two-dimensional (2D) data depending on the skin condition of the user. That is, when scanning the skin, the imaging device combines (arrays) two-dimensional (2D) pixel data and outputs the data as a skin image.

The measurer is provided with a plurality of imaging devices configured to allow each imaging device to take a necessary image of a part of the user's skin, and to image a 3D slide of the user while moving along a rail (not shown) inside the housing.

Here, the imaging device may further include a sensor such as a fingerprint recognition sensor, and the sensor may be a skin scanner. Of course, the skin image may be acquired using an imaging device such as a camera.

The measurer may acquire a skin image from human skin using the imaging device. In particular, the measurer may acquire an image of the skin of a face or an image of the skin of a part of the face.

In more detail, the measurer may represent a region of the skin image in which the skin contains a great amount of moisture in a dark shade in the skin image. Accordingly, the dark part of the skin image is determined to indicate a low moisture amount, and the moisture content of the user's skin can thus be detected based on the image.

In addition, the degree of pigmentation and the location of erythema may be determined based on the user's skin image acquired using the imaging device.

In addition, wrinkles located on the skin of the user may be measured. By measuring the location of the wrinkles, the depth, length and number of wrinkles can be detected. More preferably, the depth of the wrinkles may be detected using a sensor.

In an embodiment of the present invention, the imaging device is configured to receive an image of the skin, calculate a pixel value for the received image, and receive data on skin moisture content, skin pigmentation, skin erythema, and skin wrinkles.

That is, the measurer is configured to determine the brightness or color coordinates corresponding to the pixel value of the image and calculate data of the moisture content, skin pigmentation, skin erythema and skin wrinkles.

More preferably, in an embodiment of the present invention, the measurer may represent a pixel value of an effective skin image within a range of 0 to 255. In addition, as the pixel value of the effective skin image approaches 0, darkness increases, and the moisture content of the skin is measured based thereon. In an embodiment of the present invention, as the number of pixel values that reach 0 increases, the moisture content of the skin decreases.

In another embodiment of the present invention, the measurer may measure a skin impedance by measuring a voltage change according to a skin condition when passing a micro current through the skin, and the moisture content of the skin may be detected based on the skin impedance.

The measurer of the present invention may measure oil content using a method similar to that for the moisture content, and the measurement method is not limited.

In an embodiment of the present invention, a method of evaluating the elasticity of the dermal layer of the skin using negative pressure may be used in order to measure skin elasticity, and at least three measurements may be made at the same location.

However, as disclosed above, the methods for measuring moisture content, skin barrier, skin pigmentation, skin erythema, skin wrinkles, and skin elasticity are not limited to the methods disclosed above, and may include any of all conventional methods.

The determiner of the present invention converts the measured user's oil content, moisture content, skin barrier index, skin pigmentation index, skin erythema index, skin wrinkle or skin elasticity index into an index of 1 to 10, and stores the index in the storage.

The determiner is configured to recommend a cosmetic ingredient according to a skin condition based on the calculated skin elasticity index, and is more preferably configured to provide a cosmetic containing an effective cosmetic ingredient based on the user's skin data. More preferably, the housing of the device for providing serial skin type analysis and preparing a customized cosmetic product may include a discharge port, configured to prepare a cosmetic or cosmetic ingredient determined by the determiner immediately and to package the same into a sealed container.

In an embodiment of the present invention, the determiner may execute at least one of a moisture content measurement algorithm, an erythema detection algorithm, or a wrinkle detection algorithm. For example, the wrinkle detection algorithm may be an algorithm based on a Frangi filter. The pore detection algorithm may be an algorithm using blob detection based on a luminance ratio. The erythema detection algorithm may be an algorithm that converts a skin image into HSV color space and performs quantification based on the distance.

The present invention is configured to control at least one of temperature, brightness or humidity so as to maintain a constant measurement environment inside the housing. More preferably, the device for providing serial skin type analysis and preparing a customized cosmetic product further includes a measurement state maintenance unit located inside the housing, and the measurement state maintenance unit is an element configured to maintain a constant imaging environment in the housing, and includes at least one of a temperature controller, a brightness controller or a humidity controller.

There is a fundamental problem that the image may differ depending on the conditions at the time of imaging to perform skin diagnosis. In an attempt to solve this problem, the measurement state maintenance unit may maintain predetermined conditions inside the housing every time imaging is performed.

The temperature controller maintains a constant temperature inside the housing, so as to prevent the pores from expanding or contracting due to a change in the elasticity of the skin depending on the temperature. In one embodiment, the temperature controller may include a thermometer configured to measure an internal temperature and an internal heating wire (not shown) or a ventilating fan (not shown) configured to control the internal temperature. The temperature controller may use various techniques for maintaining a constant temperature inside the housing.

The brightness controller can solve a problem of shading that may occur depending on the brightness by maintaining a constant brightness inside the housing. In one embodiment, the brightness controller may include a light source configured to emit light and a sensing unit configured to detect internal illuminance. As the light source, a surface light source may be used to solve a problem of reflected light or a shadow that may be formed when a point light source is used. The position of the surface light source is determined according to the size and shape of the housing. The sensing unit can adjust the brightness of the light by measuring the brightness of the light inside. The brightness controller may use any of various techniques generally used to maintain constant brightness inside the housing. More preferably, the brightness controller may be moved to a position corresponding to the image device in order to prevent the brightness of the skin from varying upon the movement of the imaging device.

The humidity controller maintains constant humidity inside the housing, thereby solving a problem in which the skin contracts or expands depending on the humidity. In one embodiment, the humidity controller may be provided with a humidity control device configured to control the humidity inside the housing.

As described above, the device for providing serial skin type analysis and preparing a customized cosmetic product is capable of performing imaging regardless of time and place under a constant environment and constant conditions every time imaging is performed, thus exerting effects of making it easy to perform comparative evaluation and follow-up observation.

The storage unit of the present invention is configured to store the skin image and skin condition data of the user and further to store previous data of the user, input through an input unit. Furthermore, the storage unit is configured to access the stored data from a portable terminal of the user and is configured as a memory format embedded in a server or the device for providing serial skin type analysis and preparing a customized cosmetic product.

The device for providing serial skin type analysis and preparing a customized cosmetic product according to an embodiment of the present invention includes presenting a questionnaire about a user's skin condition through a display unit or an audio output unit of the terminal of the user. In this case, the questionnaire may be stored in the storage unit of the device for providing serial skin type analysis and preparing a customized cosmetic product according to an embodiment of the present invention.

The user terminal may present the questionnaire visually or aurally, and may be a smart phone, a laptop computer, a desktop computer, or a tablet PC, but is not limited thereto. The display unit of the user terminal may include an LCD, OLED, CRT, PDP, or a beam projector, but is not limited thereto.

The device for providing serial skin type analysis and preparing a customized cosmetic product according to an embodiment of the present invention includes processing the result of a questionnaire, input through the input unit of the user terminal. The input unit of the user terminal may include a keyboard, a mouse, a stylus pen, or a touch panel, but is not limited thereto.

The device for providing serial skin type analysis and preparing a customized cosmetic product according to an embodiment of the present invention includes correlating at least one of the analysis result of the skin image or the skin impedance with the result of the questionnaire to derive the skin type of the user and a cosmetic suitable for the skin type.

That is, the determiner is configured to adjust the data of the user, measured through the measurer, in response to the result of questionnaire input through the input unit, a preliminary questionnaire, or the like. More preferably, the questionnaire information input through the input unit may include skin care interests, age, and previous care methods of a user.

Moreover, the device for providing serial skin type analysis and preparing a customized cosmetic product according to an embodiment of the present invention transmits and receives commands including messages, data, information, and one or more programs (i.e., application code) through a communication link and a transmitter.

The transmitter may include a short-range communication module such as a Bluetooth or NFC module, but is not limited thereto. In addition, the transmitter may access a network, and the network may include at least one of a LAN, WLAN, PSTN, or a cellular phone network, but is not limited thereto.

The transmitter may include at least one of a network interface or a mobile communication module configured to access the network. The mobile communication module can be connected to a mobile communication network for a corresponding generation (for example, a 2G to 5G mobile communication network).

As described above, the device for providing serial skin type analysis and preparing a customized cosmetic product according to the present invention measures a skin condition of a user, indexes data of the measured skin condition, and diagnoses the skin condition based on the index, thereby presenting cosmetic ingredients suitable for the user's skin condition.

As apparent from the foregoing, the present invention can exhibit the following effects based on the configuration, combination, and use relationship with the embodiments described above.

The present invention can provide a comprehensive skin condition and thus has an effect of facilitating the user's aftercare.

In addition, the present invention has effects of providing a device for providing serial skin type analysis and preparing a customized cosmetic product that is capable of measuring a skin condition, and at the same time, producing a cosmetic product.

The detailed description above is merely illustrative of the present invention. That is, the above description illustrates preferred embodiments of the present invention, and the present invention can be used in various other combinations, modifications and environments. That is, alterations or modifications are possible within the scope of the concept of the invention disclosed in the present specification, the scope equivalent to the disclosed content, and/or the skill or knowledge of the art. The described embodiment is the best embodiment for implementing the technical idea of the present invention, but various modifications required in specific application fields and uses of the present invention are also possible. Therefore, the detailed description of the invention should be not construed as limiting the present invention to the disclosed embodiment. In addition, the appended claims should be construed as including other embodiments.

What is claimed is:

1. A device for providing serial skin type analysis and preparing a customized cosmetic product comprising:
a measurer configured to measure a skin condition of a user;
a storage unit configured to store data of the skin condition measured by the measurer;
a determiner configured to index the measured skin condition data and determine an effective cosmetic ingredient based on the skin condition; and
a transmitter configured to transmit the skin condition data.

2. The device according to claim 1, wherein the measurer is configured to measure at least one of skin moisture content, skin barrier, skin pigmentation, skin erythema, skin wrinkles, or skin elasticity of a user.

3. The device according to claim 2, wherein the determiner is configured to create an index of data of the at least one of moisture content, skin barrier, skin pigmentation, skin erythema, skin wrinkles or skin elasticity of the user, measured by the measurer, and to store the index in the storage unit.

4. The device according to claim 3, wherein data on each of the measured moisture content, skin barrier, skin pigmentation, skin erythema, skin wrinkles, and skin elasticity of the user is converted to an index of 1 to 10 and is stored in the storage unit.

5. The device according to claim 1, wherein the storage unit assigns weights to the skin condition data of the user, measured by the measurer, based on an item input by the user.

6. The device according to claim 1, wherein the measurer is configured to measure at least one of a temperature, thickness, color, or wrinkle depth of a skin of the user.

7. The device according to claim 1, further comprising a display unit configured to display the measured skin condition data.

8. The device according to claim 1, wherein the storage unit is configured to store the skin condition data of the user and calculate means of oil content, moisture content, skin barrier index, skin pigmentation index, skin erythema index, skin wrinkle index, and skin elasticity index based on accumulated information.

9. The device according to claim 1, wherein the determiner is configured to adjust skin data of the user, measured by the measurer, based on previous information input by the user.

10. The device according to claim 1, further comprising a measurement state maintenance unit configured to maintain a temperature, a humidity and a brightness when the measurer measures the skin condition of the user.
